(19) United States
(12) Patent Application Publication
(10) Pub. No.: US 2009/0170734 A1
(43) Pub. Date: Jul. 2, 2009

(54) NOVEL IMIDAZOLINIUM SALTS WITH LOW MELTING POINT, PROCESSES FOR PREPARATION THEREOF AND USE THEREOF AS A LUBRICANT

(76) Inventors: Peter SCHWAB, Essen (DE); Hans-Juergen Koehle, Mainhausen (DE); Thomas Salomon, Bad Soden-Salmuenster (DE); Kerstin Hell, Essen (DE)

Correspondence Address: FROMMER LAWRENCE & HAUG, 745 FIFTH AVENUE- 10TH FL., NEW YORK, NY, 10151, US

(21) Appl. No.: 12/337,906

(22) Filed: Dec. 18, 2008

(30) Foreign Application Priority Data
Dec. 29, 2007 (DE) 102007063149.0

(51) Int. Cl. C10M 133/46; C07D 233/04; C07D 403/02

(52) U.S. Cl. 508/283; 548/350.1; 548/313.7

(57) ABSTRACT

The invention relates to novel imidazolinium salts, to processes for preparation thereof and to the use of the imidazolinium salts or of a mixture of imidazolinium salts with further (lubricating) substances as lubricants.

This application claims benefit under 35 U.S.C. 119(a) of German patent application 10 2007 063 149, filed on 29 Dec. 2007.

Any foregoing applications, including German patent application DE 10 2007 063 149, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

The invention relates to novel imidazolinium salts with low melting points and to a process for preparation thereof. It further relates to the use of these substances as a base oil or as an additive in lubricants.

### STATE OF THE ART

Lubricants are used for lubrication and serve to reduce friction and wear, and also for force transmission, cooling, vibration damping, sealing action and corrosion protection.

During any movement, there is relative movement between components and hence friction between the surfaces. In order to minimize energy loss and wear, it is therefore necessary to apply a lubricant between the moving components. For the lubrication, mineral oils, poly-alpha-olefins, native oils (e.g. rapeseed oils), synthetic ester oils and low-viscosity polyglycols are used.

Lubricant oils are the most important industrial lubricants. They serve to reduce friction, which causes noise and particularly material wear. Furthermore, the use of lubricant oil also enables the removal of heat. Lubricant oil forms a lubricating film between moving surfaces, for instance in a hinge.

The first synthetic lubricants were developed in the 1930s and 1940s. The starting points were tests with mineral oil and various additives, and later synthesis by means of ethylene. One way in which these synthetic lubricant oils were significant was the change and dependence of the viscosity even under extreme temperature conditions. These studies led to the preparation of more than 3500 esters in the years mentioned, also including diesters and polyol esters.

Modern four-stroke motor oils are generally mineral or synthetic base oils comprising an additive package. The base oil in the case of mineral oil is a mineral oil distillate. The distillates are then also filtered, clarified and refined, among other processes, so as to obtain an unalloyed oil with a particular viscosity. This oil is not a pure substance, but rather a fraction, i.e. a mixture of different hydrocarbons with a similar boiling range. For a long time, these oils were the only oils used in motor vehicles; they are still supplied today by some manufacturers as compressor oil or machine oil (very old engines (pre-war machines) require these oils, since their seal materials are often incompatible with the modern additives).

Until the 1940s and thereafter, high-load engines (in particular motorcycle racing engines) were also lubricated with vegetable oil (castor oil).

Even in the past, various agents which were intended to improve the oil quality of the mineral oils were supplied; some of them are still available today. In addition to solid-state additives such as molybdenum disulphide and colloidal graphite (spherical graphite), there were also various chemical additives, some of which fulfilled their purpose quite efficiently. In about the 1940s, oils equipped with such additives as standard came onto the market, and were marketed as HD (heavy duty) oils.

Internal combustion engines make high demands on the motor oil. The motor oil is not only a lubricant, but also has further important engine functions, specifically:
transmission of forces (hydraulically in chain tensioners and tappets),
wear protection (of the engine parts moving against one another),
corrosion protection of the engine parts against aggressive combustion products by formation of protective layers on the metal surface,
sealing (of the combustion chamber from the crankcase, of the intake and exhaust gas channels through the valve guides to the valve train),
cooling (of piston and crankshaft in particular),
neutralization of acidic combustion products by chemical conversion,
keeping engine parts clean by detaching combustion residues (and ageing products of the motor oil) with oil-soluble soaps,
physical lubrication (separation of metal surfaces moving against one another),
dispersion of solid extraneous substances, dust, attritus, combustion products such as soot or ash.

In order to be able to fulfil these functions, many different requirements are made on the motor oil, which are characterized by chemical, physical and technological properties. These properties are, in simplified terms:
viscosity and flow behaviour,
surfaceactive behaviour,
neutralization capacity,

In addition, the following demands are made on the motor oil:
neutral behaviour towards seal materials,
low foaming tendency,
long use time, long oil change intervals,
low oil consumption,
low fuel consumption,
fuel compatibility,
environmental compatibility.

These known lubricants each have different disadvantages. Native oils have considerable deficiencies in the cold performance, in the UV stability, in the ageing behaviour, and in the thermal stability and the water resistance. Mineral oils have low viscosity indices, high evaporation losses, low cold suitability and moderate thermal durability. Polyglycols exhibit a low thermal stability, a high evaporation loss, low viscosities and poor seal compatibilities. Ester oils are not hydrolysisstable and exhibit low shear stabilities. All abovementioned lubricants are charged statically in the course of the filtration process, especially at low temperatures, which can lead to dangerous electrical discharges.

In the selection of the lubricants, it should generally be ensured that the substance has very low coefficients of friction, sufficient cold flow capacity (down to −60° C.), good thermal stability (to 400° C.), good paint and seal compatibility, low toxicity and ecotoxicity, high shear stability, high viscosity indices, low corrosivity, good additizability and very good wear protection.

### OBJECTIVE

It is therefore an object of the invention to provide a lubricant for the lubrication of moving parts which offers an improved profile of properties, in order, especially over a wide temperature range, to enable a very low friction loss (fuel efficiency).

This is achieved in accordance with the invention by the use of at least one novel imidazolinium salt or of a mixture of novel imidazolinium salts or of an addition of novel imidazolinium salts to other lubricants.

The invention therefore provides novel 1-hydroxyalkylimidazolinium and 1-alkoxyalkylimidazolinium salts of the general formula (I)

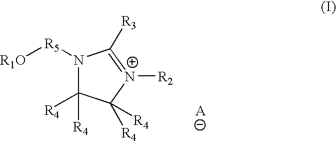

where
R₁ is hydrogen or an optionally branched, optionally unsaturated alkyl radical which optionally contains oxygen atoms or nitrogen atoms and has 1 to 20 carbon atoms, preferably 1 to 12 carbon atoms, more preferably 1 to 8 carbon atoms, most preferably 1 to 4 carbon atoms
R₂ is hydrogen or an optionally branched, optionally unsaturated alkyl radical which optionally contains oxygen atoms or nitrogen atoms and has 1 to 20 carbon atoms, preferably 1 to 12 carbon atoms, more preferably 1 to 4 carbon atoms,
R₃ is an optionally branched, optionally unsaturated alkyl radical which is optionally interrupted by oxygen atoms and has 1 to 20 carbon atoms, preferably 1 to 12 carbon atoms, more preferably 1 to 8 carbon atoms, most preferably 1 to 4 carbon atoms
R₄ are each independently hydrogen or alkyl, preferably butyl, propyl, ethyl, methyl or hydrogen,
R₅ is a divalent saturated or unsaturated, straight-chain, branched or cyclic hydrocarbon radical optionally interrupted by oxygen or nitrogen atoms or carboxyl groups and optionally substituted, preferably butylene, propylene, ethylene, methylene, more preferably butylene, and
A is a counterion to the positive charges on the quaternary nitrogen groups,
   and 1,3-dihydroxyalkylimidazolinium and 1-alkoxyalkyl-3-hydroxyimidazolinium salts correspond to the general formula (II)

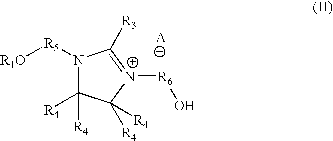

where
R₁, R₃, R₄ and R₅ are each as defined under formula (I) and
R₆ is a divalent, saturated or unsaturated, straight-chain, branched or cyclic hydrocarbon radical optionally interrupted by oxygen or nitrogen atoms or carboxyl groups and optionally substituted, preferably butylene, propylene, methylene, more preferably butylene or ethylene, and
A is a counterion to the positive charges on the quaternary nitrogen groups,
   and
   to the general formula (III)

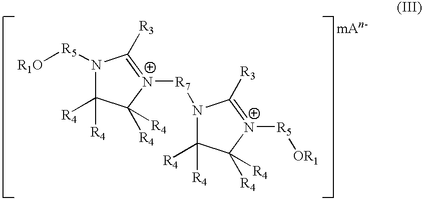

where
R₁, R₃, R₄ and R₅ are each as defined under formula (I) and
R₇ is a divalent, saturated or unsaturated, straight-chain, branched or cyclic hydrocarbon radical optionally interrupted by oxygen or nitrogen atoms or carboxyl groups and optionally substituted, preferably butylene, propylene, ethylene, methylene, more preferably butylene or ethylene,
A is a counterion to the positive charges on the quaternary nitrogen groups and the multiple of m and n corresponds to the charge of the imidazolinium cation; m and/or n is preferably 1 or 2.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

The novel imidazolinium salts have, as anions [A]ⁿ⁻, preferably those which satisfy the demanding field of use as lubricants and have, for example, a high thermal resistance and thus adversely affect neither the viscosity properties nor the melting properties of the salts, for example selected from the groups of the halides, carboxylates, phosphates, thiocyanates, isothiocyanates, dicyanamides, sulphates, alkylsulphates, sulphonates, alkylsulphonates, tetrafluoroborates, hexafluorophosphates or else bis(trifluoromethyl-sulphonyl)imides, the aforementioned anions constituting a small selection from the large number of possible anions, and hence there being no intention to make any claim to completeness or even impose a restriction.

The imidazolinium salts used with preference in accordance with the invention consist of at least one of the aforementioned cations of the formula (I), (II) or (III) combined with in each case at least one anion [A]ⁿ⁻.

A common feature of the imidazolinium salts of the formulae (I), (II) and (III) is the imidazolinium skeleton, which has a significant effect on the product properties with regard to the end use as a lubricant or lubricant constituent. Whether the imidazolinium skeleton has different substitution or is even present in the form of a dimeric structure or even oligomeric form appears to play a minor role. Important factors for the product properties are, for example, the melting points or melting ranges of the compounds, the viscosity-temperature behaviour thereof over a wide temperature range, their low coefficients of friction, their thermal stability with respect to decomposition and their balanced hydrophilicity, which indicates improved handling as compared with the market-leading lubricants which are usually hydrophobic.

The anion [A]ⁿ⁻ of the imidazolinium salt is, for example, selected from:
the group of the halides, pseudohalides, mineral acid anions and halogenated compounds of the formulae: F⁻ Cl⁻, Br⁻, I⁻, BF₄⁻, PF₆⁻, BCl₄⁻, CF₃SO₃⁻, (CF₃SO₃)₂N⁻, CF₃CO₂⁻, CCl₃CO₂⁻, CN⁻, SCN⁻, OCN⁻, NO₂⁻, NO₃⁻, N(CN)⁻;
the group of the sulphates, sulphites and sulphonates of the general formulae: SO₄²⁻, HSO₄⁻, SO₃^{2″}, HSO₃⁻, R^{a}OSO₃⁻, R^{a}SO₃⁻;
the group of the phosphates of the general formulae: PO₄³⁻, HPO₄²⁻, H₂PO⁴⁻, R^{a}PO₄²⁻, HR^{a}PO₄—, R^{a}R^{b}PO₄⁻;
the group of the phosphonates and phosphinates of the general formula: R^{a}HPO₃⁻, R^{a}R^{b}PO₂⁻, R^{a}R^{b}PO₃⁻;
the group of the phosphites of the general formulae: PO₃³⁻, HPO₃²⁻, H₂PO₃⁻, R^{a}PO₃²⁻, R^{a}HPO₃⁻, R^{a}R^{b}PO₃⁻;
the group of the phosphonites and phosphinites of the general formula: R^{a}R^{b}PO₂⁻, R^{a}HPO₂⁻, R^{a}R^{b}PO⁻, R^{a}HPO⁻;
the group of the carboxylates of the general formulae: R^{a}COO⁻;
the group of the borates of the general formulae: BO₃³⁻, HBO₃²⁻, H₂BO₃⁻, R^{a}R^{b}BO₃⁻, R^{a}HBO₃⁻, R^{a}BO₃⁻, B(OR^{a})(OR^{b})(OR^{c})(OR^{d})⁻, B(HSO₄)⁻, B(R^{a}SO₄)⁻;
the group of the boronates of the general formulae: R^{a-}BO₂²⁻, R^{a}R^{b}BO⁻;
the group of the carbonates and carbonic esters of the general formulae: HCO₃⁻, CO₃²⁻, R^{a}CO₃⁻;
the group of the silicates and silicic esters of the general formulae: SiO₄⁴⁻, HSiO₄³⁻, H₂SiO₄²⁻, H₃SiO₄⁻, R^{a}SiO₄³⁻, R^{a}R^{b}SiO₄²⁻, R^{a}R^{b}R^{c}SiO₄⁻, HR^{a}SiO₄²⁻, H₂R^{a}SiO₄⁻, HR^{a}R^{b}SiO₄⁻;
the group of the alkyl- and arylsilane salts of the general formulae: R^{a}SiO₃³⁻, R^{a}R^{b}SiO₂²⁻, R^{a}R^{b}R^{c}SiO⁻, R^{a}R^{b}R^{c}SiO₃⁻, R^{a}R^{b}R^{c}SiO₂⁻, R^{a}R^{b}SiO₃²⁻;
the group of the carboximides, bis(sulphonyl)imides and sulphonylimides of the general formulae:

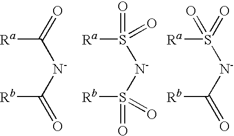

the group of the methides of the general formula:

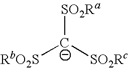

the group of the alkoxides and aryl oxides of the general formulae: R^{a}O⁻;
the group of the halometallates of the general formula [MᵣHalₜ]^{s-} where M is a metal and Hal is fluorine, chlorine, bromine or iodine, r and t are each positive integers and specify the stoichiometry of the complex, and s is a positive integer and specifies the charge of the complex; for example AlCl₄⁻, Al₂Cl₇⁻, Al₃Cl₁₀⁻, AlBr₄⁻, FeCI₄⁻, SbF₆⁻, AsF₆⁻, ZnCl₃⁻, SnCl₃⁻, CuCl₂⁻,
the group of the sulphides, hydrogensulphides, polysulphides, hydrogenpolysulphides and thiolates of the general formulae:
   S²⁻, HS⁻, [Sᵥ]²⁻, [HS_{V}]⁻, [R^{a}S]⁻, where v is a positive integer from 2 to 10;
the group of the complex metal ions such as Fe(CN)₆³⁻, Fe(CN)₆⁴⁻, MnO₄⁻, Fe(CO)₄⁻.

In these formulae, R^{a}, R^{b}, R^{c} and R^{d} are each independently
hydrogen;
C₁-C₃₀-alkyl and the aryl-, heteroaryl-, cycloalkyl-, halogen-, hydroxyl-, amino-, carboxyl-, formyl-, —O—, —CO—, —CO—O— or —CO—N<-substituted components thereof, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, tetracosyl, pentacosyl, hexacosyl, heptacosyl, octacosyl, nonacosyl, triacontyl, phenylmethyl (benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, 3-cyclohexylpropyl, methoxy, ethoxy, formyl, acetyl or C_{q}F_{2(q-a)+(1-b)}H_{2a+b} where q<30, 0≦a≦q and b=0 or 1 (for example CF₃, C₂F₅, CH₂CH₂—C_{(q-2)}F_{2(q-2)+1}, C₆F₁₃, C₈F₁₇, C₁₀F₂₁, C₁₂F₂₅);
C₃-C₁₂-cycloalkyl and the aryl-, heteroaryl-, cycloalkyl-, halogen-, hydroxyl-, amino-, carboxyl-, formyl-, —O—, —CO— or —CO—O-substituted components thereof, for example cyclopentyl, 2-methyl-1-cyclopentyl, 3-methyl-1-cyclopentyl, cyclohexyl, 2-methyl-1-cyclohexyl, 3-methyl-1-cyclohexyl, 4-methyl-1-cyclohexyl or C_{q}F_{2(q-a)-(1-b)}H_{2a-b} where q≦30, 0≦a≦q and b=0 or 1;
C₂-C₃₀-alkenyl and the aryl-, heteroaryl-, cycloalkyl-, halogen-, hydroxyl-, amino-, carboxyl-, formyl-, —O—, —CO— or —CO—O-substituted components thereof, for example 2-propenyl, 3-butenyl, cis-2-butenyl, trans-2-butenyl or C_{q}F_{2(q-a)-(1-b)}H_{2a-b} where q≦30, 0≦a≦q and b=0 or 1;
C₃-C₁₂-cycloalkenyl and the aryl-, heteroaryl-, cycloalkyl-, halogen-, hydroxyl-, amino-, carboxyl-, formyl-, —O—, —CO— or —CO—O— substituted components thereof, for example 3-cyclopentenyl, 2-cyclohexenyl, 3-cyclohexenyl, 2,5-cyclohexadienyl or C_{q}F_{2(q-a)-3(1-b)}H_{2a-3b} where q≦30, 0≦a≦q and b=0 or 1;
aryl or heteroaryl having 2 to 30 carbon atoms and the alkyl-, aryl-, heteroaryl-, cycloalkyl-, halogen-, hydroxyl-, amino-, carboxyl-, formyl-, —O—, —CO— or —CO—O-substituted components thereof, for example phenyl, 2-methylphenyl (2-tolyl), 3-methylphenyl (3-tolyl), 4-methylphenyl, 2-ethylphenyl, 3-ethylphenyl, 4-ethylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 4-phenylphenyl, 1-naphthyl, 2-naphthyl, 1-pyrrolyl, 2-pyrrolyl, 3-pyrrolyl, 2-pyridinyl, 3-pyridinyl, 4-pyridinyl or C₆F₍₅₋ₐ₎Hₐ where 0≦a≦5; or
two radicals are an unsaturated, saturated or aromatic ring optionally substituted by functional groups, aryl, alkyl, aryloxy, alkyloxy, halogen, heteroatoms and/or heterocycles and optionally interrupted by one or more oxygen and/or sulphur atoms and/or one or more substituted or unsubstituted imino groups.

Very particularly preferred anions are, for example, chloride; bromide; iodide; thiocyanate; hexafluorophosphate; trifluoromethanesulphonate; methanesulphonate; formate; acetate; glycolate; lactate; mandelate; nitrate; nitrite; trifluoroacetate; sulphate; hydrogensulphate; methylsulphate; ethylsulphate; 1-propylsulphate; 1-butylsulphate; 1-hexylsulphate; 1-octylsulphate; phosphate; dihydrogenphosphate; hydrogenphosphate; C₁-C₄-dialkylphosphates; propionate; tetrachloroaluminate; Al₂Cl₇⁻; chlorozincate; chloroferrate; bis(trifluoromethylsulphonyl)imide; bis(pentafluoroethylsulphonyl)imide; bis(methylsulphonyl)imide; bis(p-tolylsulphonyl)imide; tris(trifluoromethylsulphonyl)methide; bis(pentafluoroethylsulphonyl)methide; p-tolylsulphonate; tetracarbonylcobaltate; dimethylene glycol monomethyl ether sulphate; oleate; stearate; acrylate; methacrylate; maleate; hydrogencitrate; vinylphosphonate; bis(pentafluoroethyl)phosphinate; borates such as bis[salicylato(2-)]borate, bis[oxalato(2-)]borate, bis[1,2-benzoldiolato(2-)-O,O′]borate, tetracyanoborate, tetrafluoroborate; dicyanamide; tris(pentafluoroethyl)trifluorophosphate; tris(heptafluoropropyl)trifluorophosphate, cyclic aryl phosphates such as pyrocatecholphosphate (C₆H₄O₂)P(O)O⁻ and chlorocobaltate.

Preferred anions are selected from the group—without any claim to completeness—of the halides, bis(perfluoroalkylsulphonyl)amides or -imides, for example bis(trifluoromethylsulphonyl)imide, alkyl- and aryltosylates, perfluoroalkyltosylates, nitrate, sulphate, hydrogensulphate, alkyl- and arylsulphates, polyethersulphates and -sulphonates, perfluoroalkylsulphates, sulphonate, alkyl- and arylsulphonates, perfluorinated alkyl- and arylsulphonates, alkyl- and arylcarboxylates, perfluoroalkylcarboxylates, perchlorate, tetrachloroaluminate, saccharinate. In addition, dicyanamide, thiocyanate, isothiocyanate, tetraphenylborate, tetrakis(pentafluorophenyl)borate, tetrafluoroborate, hexafluorophosphate, polyetherphosphate and phosphate are preferred anions.

Very particularly preferred anions are: hydrogensulphate, tetrachloroaluminate, thiocyanate, methylsulphate, ethylsulphate, methanesulphonate, formate, acetate, glycolate, lactate, dimethylphosphate, diethylphosphate, p-tolylsulphonate, tetrafluoroborate and hexafluorophosphate, and also chloride, bromide.

The desired anion can be introduced by ion exchange on known ion exchangers/exchange resins.

According to the invention, in a further preferred embodiment, imidazolinium salts or mixtures thereof which comprise bis(trifluoromethylsulphonyl)imide, perfluoroalkyltosylates, alkylsulphates and -sulphonates, perfluorinated alkylsulphonates and -sulphates, perfluoroalkylcarboxylates, perchlorate, dicyanamide, thiocyanate, isothiocyanate, tetraphenylborate, tetrakis(pentafluorophenyl)borate, tetrafluoroborate, hexafluorophosphate are used.

According to the invention, in a further preferred embodiment, imidazolinium salts or mixtures thereof which are halide-free are used. Halide-free within the understanding of the present invention means a halide (counted as anion) content of less than 1 mass percent, preferably less than 0.5 mass percent and most preferably less than 0.05 mass percent.

This invention further provides a process for preparing the inventive products. This is characterized in that carboxylic acids or carboxylic acid derivatives, for example carboxylic esters, are reacted with aminoethylethanolamine with elimination of water to give the corresponding amide amines and then cyclized at further elevated temperature relative to the first process step to give the imidazoline derivative. With or without further workup, the imidazoline derivatives are then converted by alkylation to the inventive imidazolinium salts. For the alkylation, it is possible to use all alkylating agents known to those skilled in the art, especially alkyl halides, dialkyl sulphates, alkylsulphonic esters and dialkyl carbonates. The inventive substances of the formula II are quaternized by reaction with ethylene oxide/Brønsted acid. Ion exchange, for example on ion exchange resins, can be used to obtain any desired anion as the counterion.

Before the alkylation, the imidazoline derivatives can be functionalized on the free hydroxyl group. All chemical conversions which do not impair the subsequent alkylation step are possible in principle. The hydroxyl group can preferably be alkoxylated, esterified or amidated.

The inventive use of at least one imidazolinium salt provides a novel lubricant which features an outstanding profile of properties, more particularly very low coefficients of friction.

At the same time, the substances possess a profile of properties adjustable within wide ranges with regard to viscosity, density, thermal stability, anti-corrosive properties, oxidation stability, material compatibility, wear protection, cold suitability, VT behaviour (viscosity-temperature behaviour), miscibilities, hydrolysis stabilities, toxicity and ecotoxicity. On this basis it is possible to provide, for any application, a tailored lubricant which fully satisfies the requirements with regard to the abovementioned properties. It is thus possible with an imidazolinium salt or a mixture of imidazolinium salts as lubricants to achieve a high energy efficiency (“fuel efficiency”).

Owing to the fact that some imidazolinium salts, as a result of their profile of properties, can be selected such that they are resistant to high temperatures, are non-combustible and are corrosion-inhibiting, and, in relation to viscosity, density, oxidation stability, material compatibility, wear protection, cold suitability, V-T behaviour, miscibilities and hydrolysis stabilities, can be adjusted exactly to the particular specifications, these imidazolinium salts can be used advantageously as lubricants. Moreover, imidazolinium salts do not exhibit any vapour pressure below their decomposition temperature and possess intrinsic electrical conductivity.

In addition, particular preference is given to selecting those imidazolinium salts which are biodegradable and simultaneously non-toxic. As well as the aforementioned advantages, specifically these two additional properties are important criteria in the selection of lubricants for serial application in the industrial sector.

Illustrative representatives of these novel compounds are the imidazolinium salts (1) to (5) used in the examples:

imidazolinium salt (1): 1-hydroxyethyl-2,3-dimethylimidazolinium methylsulphate, melting point <25° C., decomposition temperature >200° C.
imidazolinium salt (2): 1-hydroxyethyl-2,3-diethylimidazolinium ethylsulphate, melting point <25° C., decomposition temperature >200° C.
imidazolinium salt (3): 1-hydroxyethyl-2-capryl-3-methylimidazolinium methanesulphonate, melting point <25° C., decomposition temperature >250° C.
imidazolinium salt (4): 1-hydroxyethyl-2,3-dimethylimidazolinium dicyanamide, melting point <25° C., decomposition temperature >250° C.
imidazolinium salt (5): 1,3-dihydroxyethyl-2-methylimidazolinium acetate, melting point <25° C., decomposition temperature >250° C.

Compared to the existing lubricants, the imidazolinium salts possess very low coefficients of friction over a very wide temperature range, which leads to high energy efficiency in the lubrication of components. Moreover, the viscosity of the imidazolinium salts can be adjusted to the profile of requirements. Furthermore, the imidazolinium salts or the mixture of imidazolinium salts have significantly improved additizability, especially with EP additives for further optimization of the specific friction- and wear-reducing properties.

The selection of the imidazolinium salts or of the mixture of imidazolinium salts is guided by the profile of requirements of the selected tribological application.

In a preferred embodiment of the invention, the lubricated unit is an internal combustion engine. Specifically in this process, the imidazolinium salt or the mixture of imidazolinium salts with its profile of properties as outlined above can achieve a great improvement and simplification.

To establish the particular profile of properties, the imidazolinium salt or the mixture of imidazolinium salts can be mixed with further substances, especially other salts. Preferred salts are organic salts; particular preference is given to ammonium salts and imidazolium salts.

To establish the particular profile of properties, the imidazolinium salt or the mixture of imidazolinium salts can be mixed with further substances, especially other lubricants. Preferred lubricants are mineral oils, poly-alpha-olefins, ester oils and natural oils.

To establish the particular profile of properties, the imidazolinium salt or the mixture of imidazolinium salts or the mixture of imidazolinium salts with further (lubricant) substances can be formulated with further substances. Preferred additives are corrosion inhibitors, EP (extreme pressure) additives, viscosity index improvers, antioxidants and detergents.

In addition, the particular profile of properties can be established by formulating lubricants with the imidazolinium salt or the mixture of imidazolinium salts.

In the case of use in an aqueous medium, the imidazolinium salt or the mixture of imidazolinium salts or the mixture of imidazolinium salts with further (lubricant) substances can be stabilized by adding a buffer.

In a further configuration of the present invention, the imidazolinium salt or the mixture of imidazolinium salts or the mixture of imidazolinium salts with further (lubricant) substances has a melting point below 100° C., preferably of below 50° C. and more preferably of below 25° C.

The inventive imidazolinium salts themselves already possess melting points below 150° C., preferably below 100° C. and more preferably below 25° C. For most of the inventive imidazolinium salts the melting point is between −50° C. to +150° C., preferably between −50 and +50° C. and most preferably −50° C. to 0° C. In the application as present the termology melting point is be be regarded as equivalent to pour point (pour point definition is a droplet which will no longer drop down from a capillary outlet when the temperature is lowered).

More preferably, the imidazolinium salt or the mixture of imidazolinium salts or the mixture of imidazolinium salts with further (lubricant) substances may have a fluidity range from −85° C. to 400° C., preferably from −70° C. to 300° C. and more preferably from −60° C. to 200° C. Fluidity range means the temperature range where a liquid compound is in the liquid phase (for water: e.g. 0° C. to 100° C. freezing point to boiling point).

A further preferred embodiment of the invention envisages the use of an imidazolinium salt or of a mixture of imidazolinium salts or the mixture of imidazolinium salts with further (lubricant) substances having a pour point of up to −65° C.

The imidazolinium salts or the mixture of imidazolinium salts or the mixture of imidazolinium salts with further (lubricant) substances are used in mixtures in which the imidazolinium salt or mixture thereof makes up 0.1 to 99.98% by weight of the overall mixture, preferably 75 to 99.95% by weight, more preferably 85 to 99.9% by weight.

In summary, accordingly, use of at least one imidazolinium salt or of mixtures of imidazolinium salts or the mixture of imidazolinium salts with further (lubricant) substances for the lubrication of components is proposed, on the basis of which a considerable simplification and advantages in handling are achieved. For example, lubricant leaks onto textiles or in the engine space can be washed out and off in a simple manner by water.

With regard to the fact that the imidazolinium salts of the formula (I), (II) or (III), by virtue of their adjustable profile of properties and miscibility, are resistant to high temperatures, non-combustible, corrosion-inhibiting, oxidation-stable, wear-inhibiting, shear-stable and additizable, they can be used advantageously as lubricants, especially in the internal combustion engine. Moreover, imidazolinium salts do not exhibit any vapour pressure below the decomposition temperature, can be biodegraded and are non-toxic.

As compared with the imidazolinium salts of the formula (I), (II) or (III), the common prior art lubricants, for example poly-alpha-olefins (PAOs) or mineral oils (e.g. Castrol®, trademark of Castrol Ltd.) exhibit higher friction values, a lower decomposition temperature, high flammability and high combustibility, and also poor environmental compatibility.

Owing to their profile of properties, selected imidazolinium salts are suitable for applications in which the substances are under high thermal stress briefly or for long periods. Preferred imidazolinium salts or mixtures of imidazolinium salts are notable for a thermal stability of <200° C. The thermal stability can be determined in a manner known per se.

However, thermogravimetry has been found to be particularly useful, where measurement is effected with a Shimadzu TG-50 and a heating rate of 10° C./min under a nitrogen atmosphere (nitrogen flow: 23 ml/min).

Under these conditions, thermally stable imidazolinium salts at 200° C. preferably exhibit a decrease in weight of >50%, more preferably >30%, appropriately >20%, even more preferably >10% and especially >5%, based in each case on the starting weight.

Another embodiment of the invention is directed toward improving the performance of a lubricating composition which includes but is not limited to oil compositions. In one embodiment of this aspect of the invention, the friction coefficient of a lubricating composition is lowered by adding the imidazolinium salt containing lubricating composition of the invention.

The inventive imidazolinium salts and the use thereof are described by way of example below, without any intention that the invention be restricted to these illustrative embodiments. Where ranges, general formulae or compound classes are specified hereinafter, these shall encompass not only the appropriate ranges or groups of compounds which are mentioned explicitly, but also all sub-ranges and sub-groups of compounds which can be obtained by selecting individual values (ranges) or compounds. When documents are cited in the context of the present description, their contents shall be incorporated fully into the disclosure-content of the present invention.

### WORKING EXAMPLES

In the examples adduced below, the present invention is described by way of example, without any intention that the invention, whose scope of application is evident from the whole description and the claims, be restricted to the embodiments specified in the examples.

All percentages are based on weight, unless stated otherwise.

General Preparation Method of an Imidazolinium Salt without Intermediate Isolation (Formula I)

A 4-neck glass flask is initially charged with 1.75 mol of aminoethylethanolamine (AEEA) and 0.01 mol of 30% sodium methoxide under N₂. 1 mol of the particular carboxylic ester are added at 70° C. with slight exothermicity within 1.5 h, in the course of which the temperature should not exceed 90° C. The amidation takes place within 6 to 10 h. For monitoring, an IR spectrum is recorded at 1740 nm. Sufficient alkalinity (>2 mgKOH/g) has to be ensured. On completion of amidation, the alkalinity is neutralized by means of 85% phosphoric acid and then cyclization is effected by heating to 175° C. with methanol/ethanol distillation (column). Vacuum is applied and adjusted to 60 mbar within 3 h, in the course of which the water of reaction distils. Monitoring by IR spectrum at 1600 and 1650 nm. On completion of cyclization, the excess AEEA is distilled under reduced pressure at <20 mbar and 175° C. The analytical monitoring is effected by OAN (overall amine number) and TAN (tertiary amine number). Subsequently, the mixture is cooled to 50° C. and 0.99 mol of the alkylating agent are slowly added dropwise. The progress of the reaction is monitored by amine number. After 1 to 24 h, the reaction is complete. The remaining residue can, according to the field of use, be used in accordance with the invention directly without further workup or be purified by methods known to those skilled in the art.

General Preparation Method of an Imidazolinium Salt without Intermediate Isolation (Formula II)

A 4-neck glass flask is initially charged with 1.75 mol of aminoethylethanolamine (AEEA) and 0.01 mol of 30% sodium methoxide under N₂. 1 mol of the particular carboxylic ester are added at 70° C. with slight exothermicity within 1.5 h, in the course of which the temperature should not exceed 90° C. The amidation takes place within 6 to 10 h. For monitoring, an IR spectrum is recorded at 1740 nm. Sufficient alkalinity (>2 mgKOH/g) has to be ensured. On completion of amidation, the alkalinity is neutralized by means of 85% phosphoric acid and then cyclization is effected by heating to 175° C. with methanol/ethanol distillation (column). Vacuum is applied and adjusted to 60 mbar within 3 h, in the course of which the water of reaction distils. Monitoring by IR spectrum at 1600 and 1650 nm. On completion of cyclization, the excess AEEA is distilled under reduced pressure at <20 mbar and 175° C. The analytical monitoring is effected by OAN and TAN. Subsequently, with intense cooling, 1 mol of a Brønsted acid is added sufficiently slowly that a temperature of 50° C. is not exceeded. The end of the reaction is determined by the amine number. Subsequently, the reaction vessel is evacuated and filled with nitrogen, so as to establish a pressure of 0.5 bar. After metered addition of 1 mol of ethylene oxide over 4 hours, reaction is continued at 65° C. for another 2 hours and then volatile by-products are distilled off under reduced pressure. The remaining residue can, according to the field of use, be used in accordance with the invention directly without further workup or be purified by methods known to those skilled in the art.

General Preparation Method of an Imidazolinium Salt without Intermediate Isolation (Formula III)

A 4-neck glass flask is initially charged with 1.75 mol of aminoethylethanolamine (AEEA) and 0.01 mol of 30% sodium methoxide under N₂. 1 mol of the particular carboxylic ester are added at 70° C. with slight exothermicity within 1.5 h, in the course of which the temperature should not exceed 90° C. The amidation takes place within 6 to 10 h. For monitoring, an IR spectrum is recorded at 1740 nm. Sufficient alkalinity (>2 mgKOH/g) has to be ensured. On completion of amidation, the alkalinity is neutralized by means of 85% phosphoric acid and then cyclization is effected by heating to 175° C. with methanol/ethanol distillation (column). Vacuum is applied and adjusted to 60 mbar within 3 h, in the course of which the water of reaction distils. Monitoring by IR spectrum at 1600 and 1650 nm. On completion of cyclization, the excess AEEA is distilled under reduced pressure at <20 mbar and 175° C. The analytical monitoring is effected by OAN and TAN. Subsequently, the mixture is cooled to 50° C. and 0.495 mol of a difunctional alkylating agent is slowly added dropwise. The progress of the reaction is monitored by amine number. After 1 to 24 h, the reaction is complete. The remaining residue can, according to the field of use, be used in accordance with the invention directly without further workup or be purified by methods known to those skilled in the art.

### USE EXAMPLES

The performance testing was effected with a Mini Traction Machine (MTM2) obtainable from PCS Instruments, Ltd. (London). This was used to determine the coefficients of friction at different temperatures, pressures and rotation speeds, and they were compared with products customary on the market (mineral oil, poly-alpha-olefin).

The imidazolinium salts (1) to (5) according to the above synthesis methods were tested for their suitability as lubricants. The following compounds were tested as imidazolinium salts (1) to (5):

imidazolinium salt (1): 1-hydroxyethyl-2,3-dimethylimidazolinium methylsulphate, melting point <25° C., decomposition temperature >200° C.
imidazolinium salt (2): 1-hydroxyethyl-2,3-diethylimidazolinium ethylsulphate, melting point <25° C., decomposition temperature >200° C.
imidazolinium salt (3): 1-hydroxyethyl-2-capryl-3-methylimidazolinium methanesulphonate, melting point <25° C., decomposition temperature >250° C.
imidazolinium salt (4): 1-hydroxyethyl-2,3-dimethylimidazolinium dicyanamide, melting point <25° C., decomposition temperature >250° C.
imidazolinium salt (5): 1,3-dihydroxyethyl-2-methylimidazolinium acetate, melting point <25° C., decomposition temperature >250° C.

TABLE 1
Force 75 N, speed 1000 mm/s

Coefficient of friction

| | at 40° C. | at 80° C. | at 110° C. |
|---|---|---|---|
| Imidazolinium salt (1) | very low | low | low |
| Imidazolinium salt (2) | very low | very low | low |
| Imidazolinium salt (3) | very low | low | low |
| Imidazolinium salt (4) | very low | low | low |
| Imidazolinium salt (5) | low | low | low |
| 1:1 mixture of | very low | low | low |
| imidazolinium salt (1) | | | |
| and (3) | | | |
| 1:1 mixture of | very low | very low | very low |
| imidazolinium salt (1) | | | |
| and 1-ethyl-3-methyl- | | | |
| imidazolium | | | |
| ethylsulphate | | | |
| CASTROL mineral oil | high | high | high |
| Poly-alpha-olefin PAO 8 | moderate | moderate | moderate |

TABLE 2
Force 75 N, speed 100 mm/s

Coefficient of friction

| | at 40° C. | at 80° C. | at 110° C. |
|---|---|---|---|
| Imidazolinium salt (1) | low | low | low |
| Imidazolinium salt (2) | very low | very low | low |
| Imidazolinium salt (3) | low | low | low |
| Imidazolinium salt (4) | very low | low | low |
| Imidazolinium salt (5) | low | low | low |
| 1:1 mixture of | low | low | low |
| imidazolinium salt (1) | | | |
| and (3) | | | |
| 1:1 mixture of | very low | very low | very low |
| imidazolinium salt (1) | | | |
| and 1-ethyl-3-methyl- | | | |
| imidazolium | | | |
| ethylsulphate | | | |
| CASTROL mineral oil | very high | very high | very high |
| Poly-alpha-olefin PAO 8 | high | high | high |

TABLE 3
Force 75 N, speed 10 mm/s

Coefficient of friction

| | at 40° C. | at 80° C. | at 110° C. |
|---|---|---|---|
| Imidazolinium salt (1) | low | moderate | moderate |
| Imidazolinium salt (2) | very low | very low | low |
| Imidazolinium salt (3) | low | low | low |
| Imidazolinium salt (4) | very low | low | low |
| Imidazolinium salt (5) | low | low | low |
| 1:1 mixture of | low | low | low |
| imidazolinium salt (1) | | | |
| and (3) | | | |
| 1:1 mixture of | very low | very low | very low |
| imidazolinium salt (1) | | | |
| and 1-ethyl-3-methyl- | | | |
| imidazolium | | | |
| ethylsulphate | | | |
| CASTROL mineral oil | high | very high | very high |
| Poly-alpha-olefin PAO 8 | high | high | very high |

TABLE 4
Force 40 N, speed 1000 mm/s

Coefficient of friction

| | at 40° C. | at 80° C. | at 110° C. |
|---|---|---|---|
| Imidazolinium salt (1) | very low | low | low |
| Imidazolinium salt (2) | very low | very low | low |
| Imidazolinium salt (3) | very low | low | low |
| Imidazolinium salt (4) | very low | low | low |
| Imidazolinium salt (5) | low | low | low |
| 1:1 mixture of | very low | low | low |
| imidazolinium salt (1) | | | |
| and (3) | | | |
| 1:1 mixture of | very low | very low | very low |
| imidazolinium salt (1) | | | |
| and 1-ethyl-3- | | | |
| methylimidazolium | | | |
| ethylsulphate | | | |
| CASTROL mineral oil | moderate | moderate | high |
| Poly-alpha-olefin PAO 8 | moderate | moderate | moderate |

TABLE 5
Force 40 N, speed 100 mm/s

Coefficient of friction

| | at 40° C. | at 80° C. | at 110° C. |
|---|---|---|---|
| Imidazolinium salt (1) | low | low | very low |
| Imidazolinium salt (2) | very low | very low | low |
| Imidazolinium salt (3) | very low | low | low |
| Imidazolinium salt (4) | very low | low | low |
| Imidazolinium salt (5) | low | low | low |
| 1:1 mixture of | very low | low | low |
| imidazolinium salt (1) | | | |
| and (3) | | | |
| 1:1 mixture of | very low | very low | very low |
| imidazolinium salt (1) | | | |
| and 1-ethyl-3-methyl- | | | |
| imidazolium | | | |
| ethylsulphate | | | |
| CASTROL mineral oil | moderate | moderate | high |
| Poly-alpha-olefin PAO 8 | moderate | moderate | moderate |

TABLE 6
Force 40 N, speed 10 mm/s

Coefficient of friction

| | at 40° C. | at 80° C. | at 110° C. |
|---|---|---|---|
| Imidazolinium salt (1) | low | low | low |
| Imidazolinium salt (2) | very low | very low | very low |
| Imidazolinium salt (3) | low | low | low |
| Imidazolinium salt (4) | very low | low | low |
| Imidazolinium salt (5) | low | low | low |
| 1:1 mixture of | low | low | low |
| imidazolinium salt (1) | | | |
| and (3) | | | |
| 1:1 mixture of | very low | very low | very low |
| imidazolinium salt (1) | | | |
| and 1-ethyl-3-methyl- | | | |
| imidazolium | | | |
| ethylsulphate | | | |
| CASTROL mineral oil | high | high | high |
| Poly-alpha-olefin PAO 8 | moderate | moderate | high |

The results show that the inventive imidazolinium salts have significantly lower coefficients of friction under the same test conditions as the prior art lubricants. This allows a reduction in the friction losses and an improvement in the energy balance (“fuel efficiency”).

Having thus described in detail various embodiments of the present invention, it is to be understood that the invention defined by the above paragraphs is not to be limited to particular details set forth in the above description as many apparent variations thereof are possible without departing from the spirit or scope of the present invention.

1. An imidazolinium salts of the general formulae (I), (II) or (III), where formula (I) corresponds to 1-hydroxyalkyl- and 1-alkoxyalkylimidazolinium salts 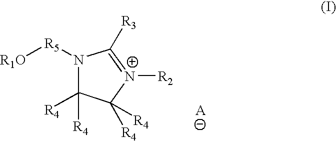
where
R₁ is hydrogen or an optionally branched, optionally unsaturated alkyl radical which optionally contains oxygen atoms or nitrogen atoms and has 1 to 20 carbon atoms,
R₂ is hydrogen or an optionally branched, optionally unsaturated alkyl radical which optionally contains oxygen atoms or nitrogen atoms and has 1 to 20 carbon atoms,
R₃ is an optionally branched, optionally unsaturated alkyl radical which is optionally interrupted by oxygen atoms and has 1 to 20 carbon atoms,
R₄ are each independently hydrogen or alkyl,
R₅ is a divalent saturated or unsaturated, straight-chain, branched or cyclic hydrocarbon radical optionally interrupted by oxygen or nitrogen atoms or carboxyl groups and optionally substituted, and
A is a counterion to the positive charges on the quaternary nitrogen groups,
and 1,3-dihydroxyalkylimidazolinium and 1-alkoxyalkyl-3-hydroxyimidazolinium salts correspond to the general formula (II) 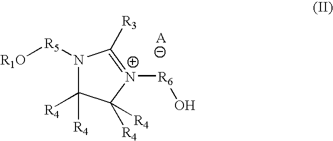
where
R₁, R₃, R₄ and R₅ are each as defined under formula (I) and
R₆ is a divalent, saturated or unsaturated, straight-chain, branched or cyclic hydrocarbon radical optionally interrupted by oxygen or nitrogen atoms or carboxyl groups and optionally substituted, and
A is a counterion to the positive charges on the quaternary nitrogen groups,
and to the general formula (III) 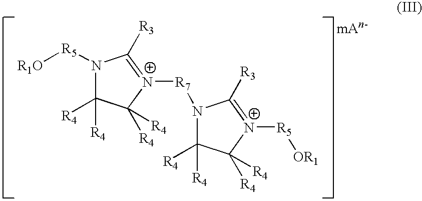
where
R₁, R₃, R₄ and R₅ are each as defined under formula (I) and
R₇ is a divalent, saturated or unsaturated, straight-chain, branched or cyclic hydrocarbon radical optionally interrupted by oxygen or nitrogen atoms or carboxyl groups and optionally substituted, A is a counterion to the positive charges on the quaternary nitrogen groups and the multiple of m and n corresponds to the charge of the imidazolinium cation.

2. The imidazolinium salts according to claim 1, wherein R₄ is butyl, propyl, ethyl, methyl or hydrogen, R₅ is butylene, propylene, ethylene, or methylene, R₆ is butylene, propylene, or methylene, and R₆ is butylene, propylene, ethylene, or methylene.

3. The imidazolinium salts according to claim 1 characterized in that R₃ is an optionally branched alkyl radical having 1 to 20 carbon atoms, R₁ is hydrogen, R₄ is hydrogen and R₂ is an optionally branched alkyl radical having 1 to 20 carbon atoms.

4. The imidazolinium salts according to claim 1 characterized in that R₃ is an optionally branched alkyl radical having 1 to 12 carbon atoms, R₁ is hydrogen, R₄ is hydrogen and R₂ is an alkyl radical having 1 to 12 carbon atoms.

5. The imidazolinium salts according to claim 3 characterized in that R₅ is butylene, R₆ is butylene or ethylene, and R₇ is butylene or ethylene.

6. The imidazolinium salts according to claim 1 characterized in that the counterion [A]ⁿ⁻ is not a halide.

7. The imidazolinium salts according to claim 1 characterized in that the melting point is below 150° C.

8. A process for preparing imidazolinium salts according to claim 1, characterized in that carboxylic acids or carboxylic acid derivatives are reacted with aminoethylethanolamine with elimination of water to give the corresponding amide amines, then cyclized at elevated temperature relative to the first process step to give the imidazoline derivative and then converted by alkylation to the inventive imidazolinium salts.

9. The process for preparing imidazolinium salts according to claim 8, characterized in that no intermediates are isolated.

10. The process for preparing imidazolinium salts according to claim 8, characterized in that the imidazoline derivative is chemically functionalized on the free hydroxyl group before the alkylation takes place.

11. The process for preparing imidazolinium salts according to claim 10, characterized in that the chemical functionalization is an alkoxylation or an esterification or an amidation.

12. The process for preparing the imidazolinium salts according to claim 10, characterized in that, for the alkylation, alkylating agents, especially alkyl halides, dialkyl sulphates, alkylsulphonic esters and dialkyl carbonates are used.

13. The process for preparing the imidazolinium salts according to claim 10, characterized in that the inventive substances are quaternized by reaction with ethylene oxide/Brønsted acid.

14. The process for preparing the imidazolinium salts according to claim 10, characterized in that the anion [A]ⁿ⁻ is obtained by ion exchange.

13. A lubrication composition comprising one or more imidazolinium salts of claim 1 optionally with further lubricating substances.

14. The lubricating composition of claim 13, wherein the further lubricating substances comprises EP additives for optimization of the friction- and wear-reducing properties.

15. The lubricating composition of claim 13, wherein the further lubricating substances comprises anticorrosives, antioxidants, viscosity index improvers, detergents, other lubricants, other salts, organic salts, ammonium salts, imidazolium salts or phosphonium salts.

16. The lubricating composition of claim 13, wherein the composition is in aqueous media and is stabilized by buffers.

17. The lubricating composition of claim 13, wherein the further lubricating substances makes up 0.1 to 99.98% by weight of the overall mixture.

18. The lubricating composition of claim 17, wherein the further lubricating substances has a fluidity range from −85° C. to 400° C.

19. A method of lowering the friction coefficient of a lubricating composition which comprises of adding one or more of the imidazolinium salts of claim 1 to said lubricating composition.

20. The method of claim 19, wherein the imidazolinium is characterized in that R₅ is butylene, R₆ is butylene or ethylene, and R₇ is butylene or ethylene.
